(19) United States
(12) Patent Application Publication
(10) Pub. No.: US 2003/0215476 A1
(43) Pub. Date: Nov. 20, 2003

(54) Multiple emulsion containing a tensioning agent

(75) Inventors: Guillaume Cassin, Villebon Sur Yvette (FR); Veronique Roger, Bagneux (FR); Veronique Chevalier, Villecresnes (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/394,160

(22) Filed: Mar. 24, 2003

Related U.S. Application Data
(60) Provisional application No. 60/374,135, filed on Apr. 22, 2002.

(30) Foreign Application Priority Data
Apr. 12, 2002 (FR) 0204609

(51) Int. Cl. A61K 7/00

(52) U.S. Cl. 424/401

(57) ABSTRACT

The present invention relates to a W/O/W multiple emulsion containing an inner aqueous phase, an oily phase and an outer aqueous phase, the emulsion containing at least one tensioning agent present at least in the inner aqueous phase of the emulsion. The invention also relates to compositions containing the emulsion and to the cosmetic use of these compositions and emulsions for, e.g., smoothing out wrinkles and fine lines and/or for restoring tautness to the skin.

### REFERENCE TO PRIOR APPLICATIONS

This application claims priority to U.S. provisional application No. 60/374,135 filed Apr. 22, 2002, and to French patent application 0204609 filed Apr. 12, 2002, both incorporated herein by reference.

### FIELD OF THE INVENTION

The present invention relates to a multiple emulsion, preferably a W/O/W multiple emulsion, comprising an inner aqueous phase, an oily phase and an outer aqueous phase, the emulsion further comprising at least one tensioning agent preferably present at least in the inner aqueous phase of the emulsion.

Preferably the tensioning agent is selected from the group consisting of: synthetic polymers, polymers of natural origin, plant proteins and protein hydrolysates; mixed silicates; wax microparticles; and colloidal particles of mineral filler.

The present invention also relates to compositions, formulations, etc., comprising this emulsion and to the cosmetic use of the multiple emulsion, compositions, and formulations for example for smoothing out wrinkles and fine lines and/or for restoring tautness to the skin.

### BACKGROUND OF THE INVENTION

In the course of the ageing process, various signs appear on the skin that are very characteristic of this ageing, reflected especially by a change in the structure and functions of the skin. The main clinical signs of ageing of the skin are especially the appearance of fine lines and deep wrinkles, which increase with age.

Disruption of the “grain” of the skin is observed in particular, ie the microrelief is less uniform and has an anisotropic nature.

It is known practice to treat these signs of ageing using cosmetic or dermatological compositions containing active agents capable of combating ageing, such as α-hydroxy acids, β-hydroxy acids and retinoids. These active agents act on wrinkles by removing the dead cells from the skin and by accelerating the process of cell renewal. However, these active agents have the drawback of being effective in treating wrinkles only after they have been applied for a certain time. It is increasingly sought to obtain an immediate effect for the active agents used.

To this end, in recent years agents with a tensioning effect have been proposed, which, immediately after application, smooth out the wrinkles and fine lines and contribute towards attenuating fatigue marks. These compounds act by forming a film that causes retraction of the stratum corneum, the superficial horny layer of the epidermis. Examples of such tensioning agents are especially dispersions of natural polymers (WO 98/29091) or of synthetic polymers, whether they are aqueous dispersions (WO 98/29092) or oily dispersions, in particular of “starburst” polymers (EP-1 043 345) or of grafted silicone polymer (EP-1 038 519). It has also been suggested to use certain mineral compounds, such as mixed silicates (EP-1 008 340) or an aqueous dispersion of colloidal particles of a mineral filler, to obtain similar effects.

However, these prior-art tensioning agents are rarely formulated in the presence of oil, in particular in O/W or W/O emulsions, but more often are in the form of sera, ie gelled aqueous compositions. The reason for this is that it is generally observed that the efficacy of these compositions is greatly reduced in the presence of oil.

However, the presence of an oily phase in a cosmetic composition is often desirable, whether to give the composition an emollient nature or to convey various liposoluble topical active agents, such as vitamins A and E, especially, which are particularly useful in anti-wrinkle compositions.

There is thus still a need to provide a composition with a tensioning effect, containing an oily phase, which is stable over time with a view to a commercial use, and which offers skin-tensioning properties that are sufficient to visibly smooth out wrinkles and fine lines as soon as it is applied.

### DETAILED DESCRIPTION OF THE PREFERRED EMBODIMENTS

This need is satisfied by the present invention by the provision of a W/O/W multiple emulsion comprising an inner aqueous phase, an oily phase and an outer aqueous phase, the emulsion further comprising at least one tensioning agent, preferably one that produces, at a concentration of 7% in water, a retraction of isolated stratum corneum of more than 1% at 30° C. under a relative humidity of 40%, wherein the tensioning agent is present at least in the inner aqueous phase of the emulsion.

The term “tensioning agent” means compounds capable of having a tensioning effect, ie which are able to make the skin taut and, as a result of this tensioning effect, to smooth out the skin and immediately reduce or eliminate the wrinkles and fine lines therein. To be most effective, the tensioning agents used according to the invention should produce, at a concentration of 7% in water, a retraction of isolated stratum corneum of more than 1% at 30° C. under a relative humidity of 40%. This retraction value is measured using a dermometer, according to the method described in Example 1 below.

The invention emulsion is suitable for further formulation in compositions and is preferably suitable for topical application to the skin, and thus preferably comprises a physiologically acceptable medium, ie a medium that is compatible with the skin and/or its integuments.

The amount of tensioning agent present in the invention emulsion may vary within a wide range depending on the desired effect, this amount being determinable by one of ordinary skill in the art based upon this disclosure. By way of example, the tensioning agent may represent from 0.01% to 10% by weight and preferably from 0.1% to 7% by weight relative to the total weight of the emulsion.

Examples of tensioning agents that are suitable for use in the present invention include: synthetic polymers, polymers of natural origin, plant proteins and protein hydrolysates; mixed silicates; wax microparticles; and colloidal particles of mineral filler.

The synthetic polymer tensioning agents may be chosen especially from: polyurethane polymers and copolymers; acrylic polymers and copolymers; sulphonated isophthalic acid polymers; and grafted silicone polymers.

The polyurethane copolymers, the acrylic copolymers and the other synthetic polymers according to the invention may be chosen especially from polycondensates, hybrid polymers and interpenetrated polymer networks (IPNs).

For the purposes of the present invention, the expression “interpenetrated polymer network” means a blend of two interlaced polymers, obtained by simultaneous polymerization and/or crosslinking of two types of monomer, the blend obtained having a single glass transition temperature.

Examples of IPNs that are suitable for use in the present invention, and also the process for preparing them, are described in U.S. Pat. Nos. 6,139,322 and 6,465,001, for example.

Preferably, the IPN according to the invention comprises at least one polyacrylic polymer and more preferably also comprises at least one polyurethane or one copolymer of vinylidene fluoride and of hexafluoropropylene.

According to one preferred form, the IPN according to the invention comprises a polyurethane polymer and a polyacrylic polymer. Such IPNs are especially those of the Hybridur series that are commercially available from the company Air Products.

An IPN that is particularly preferred is in the form of an aqueous dispersion of particles with a weight-average size of between 90 and 110 nm and a number-average size of about 80 nm. This IPN preferably has a glass transition temperature, Tg, ranging from about −60° C. to +100° C. An IPN of this type is sold especially by the company Air Products under the trade name Hybridur X-01602. Another IPN that is suitable for use in the present invention is referenced Hybridur X 18693-21.

Other IPNs that are suitable for use in the present invention comprise IPNs consisting of a blend of a polyurethane with a copolymer of vinylidene fluoride and of hexafluoropropylene. These IPNs may be prepared especially as described in patent JS-5 349 003. As a variant, they are commercially available in the form of a colloidal dispersion in water, in a ratio of the fluorinated copolymer to the acrylic polymer of between 70:30 and 75:25, under the trade names Kynar RC-10, 147 and Kynar RC-10, 151 from the company Atofina.

Examples of grafted silicone polymers are given in patent application EP-1 038 519, which is incorporated herein by reference. A preferred example of a grafted silicone polymer is polysilicone-8 (CTFA name), which is a polydimethylsiloxane onto which are grafted, via a linking chain of thiopropylene type, mixed polymer units of the poly(meth)acrylic acid type and of the polyalkyl (meth)acrylate type. A polymer of this type is available especially under the trade name VS 80 (at 10% in water) or LO 21 (in pulverulent form) from the company 3M. It is a copolymer of polydimethylsiloxane containing propylthio groups, of methyl acrylate, of methyl methacrylate and of methacrylic acid.

Polymers of natural origin useful herein include polyholosides, for example in the form of starch. They may especially be starch of natural origin, derived from rice, from corn, from potato, from cassava, from pea, from Triticum aestivum wheat, from oat, etc. Another type of polyholoside consists of carrageenans, alginates, agars, gellans, cellulose-based polymers and pectins.

The polyholoside used in the composition according to the invention will preferably be in the form of a microgel or aqueous dispersion of gel microparticles, the particles having, for example, a mean diameter of between 0.5 and 100 μm and preferably between 5 and 50 μm.

The polymer of natural origin may also be selected from the group consisting of latexes including shellac resin, sandarac gum, dammar resins, elemi gums, copal resins and cellulose-based derivatives, and mixtures thereof.

Examples of plant proteins and plant protein hydrolysates that may be used as tensioning agents according to the invention include proteins and protein hydrolysates from corn, rye, Triticum aestivum wheat, buckwheat, sesame, spelt, pea, bean, lentil, soybean and lupin.

Another class of tensioning agents that may be used according to the invention is mixed silicates. This expression means any silicate of natural or synthetic origin containing several types of cations selected from the group consisting of alkali metals (for example Na, Li or K) or alkaline-earth metals (for example Be, Mg or Ca) and transition metals.

Phyllosilicates are preferably used, ie silicates having a structure in which the SiO₄ tetrahedra are organized as leaflets between which the metal cations are enclosed.

A family of silicates that is particularly preferred as tensioning agents herein is the laponite family. Laponites are magnesium lithium sodium silicates having a layered structure similar to that of montmorillonites. Laponite is the synthetic form of the natural mineral known as “hectorite”. The laponite sold under the name Laponite XLS or Laponite XLG by the company Rockwood may be used, for example.

Yet another class of tensioning agents that may be used in the present invention is wax microparticles. These are particles with a diameter generally less than 5 μm, or better still less than 0.5 μm, and consisting essentially of a wax or a mixture of waxes chosen, for example, from carnauba wax, candelilla wax or alfalfa wax. The melting point of the wax or of the wax mixture is preferably between 50° C. and 150° C.

Also as a variant, it is possible to use as tensioning agent according to the invention colloidal particles of mineral fillers. The term “colloidal particles” means particles with a number-average diameter of between 0.1 and 100 mm and preferably between 3 and 30 mm.

Examples of mineral fillers include: silica, cerium oxide, zirconium oxide, alumina, calcium carbonate, barium sulphate, calcium sulphate, zinc oxide and titanium dioxide. A mineral filler that is particularly preferred is silica. Colloidal silica particles are especially available in the form of an aqueous dispersion from the company Catalysts & Chemicals under the trade names Cosmo S-40 and Cosmo S-50.

The emulsion according to the invention may be prepared according to any method known to those skilled in the art for preparing W/O/W emulsions and especially by emulsification of a reverse emulsion (W/O), known as the primary emulsion, in a gelled aqueous phase.

It may be a water/oil/water triple emulsion, comprising a gelled outer aqueous phase, an oily phase comprising a silicone emulsifier, chosen in particular from alkyldimethicone copolyols and dimethicone copolyols, and optionally a silicone oil, and an outer aqueous phase containing at least one emulsifying copolymer consisting of a major fraction of a C₃-C₆ monoolefinically unsaturated carboxylic acid monomer or the anhydride thereof and a minor fraction of acrylic acid fatty ester monomer, in particular an acrylate/C₁₀-C₃₀-alkylacrylate copolymer. This type of triple emulsion is described especially in patent applications EP-0 908 170 and EP-0 648 102.

As a variant, it may be a water/oil/water triple emulsion, the outer aqueous phase of which contains an acrylic or methacrylic acid polymer or copolymer combined with a polyglyceryl methacrylate, and the oily phase of which may contain a fluoro oil, as described in patent application EP-0 507 693.

As a further variant, it may be a water/oil/water emulsion, one of the aqueous phases of which has a water activity value of less than 0.85, as described in patent application EP-0 779 071.

According to one preferred embodiment of the invention, the outer aqueous phase is free of polyacrylamide-based gelling agent, in particular of polyacrylamidomethanepropanesulphonic acid, and of triethanolamine. In the case especially when the tensioning agent consists of colloidal silica particles, the inventors have in fact demonstrated that the presence of one and/or the other of these compounds has a tendency to destabilize the W/O/W multiple emulsion, leading to the production of an emulsion of the oil-in-water type and to setting of the composition after an interval of a few hours to a few days. A stabile W/O/W multiple emulsion may, however, be obtained by replacing the triethanolamine with sodium hydroxide, in the case where the presence of a neutralizer is necessary in the outer aqueous phase, for example to neutralize a carbomer.

In addition, the inventors have found that the tensioning effect of the compositions according to the invention, in particular when the tensioning agent consists of colloidal silica, is improved in the absence of low molecular weight polyol, such as glycerol. Thus, according to another preferred embodiment of the invention, the emulsion is free of glycerol and of propylene glycol.

The emulsion according to the invention is especially useful to treat the signs of ageing of the skin and in particular to smooth out or attenuate wrinkles and fine lines and/or to restore tautness to the skin.

A subject of the invention is thus also a cosmetic process for treating wrinkled skin, comprising the application to the skin of an emulsion as defined above.

A subject of the invention is also the cosmetic use of the composition as defined above to smooth out wrinkles and fine lines and/or to restore tautness to the skin, in particular of the face and/or the neck.

A further subject of the invention is a composition, preferably a cosmetic composition, comprising the invention emulsion. The invention composition can further comprise, for example, further aqueous and/or oil phases, etc., as well as physiologically acceptable media, carriers, active agents, etc. In one preferred embodiment the invention emulsion may be incorporated into an oil phase to provide a W/O/W/O composition, for example. The above uses described for the invention emulsion apply with equal force to the invention composition.

To reinforce the anti-ageing effects of the composition and/or emulsion according to the invention, it may contain, besides the tensioning agent described above, at least one compound selected from the group consisting of: desquamating and/or moisturizing agents; depigmenting or propigmenting agents; anti-glycation agents; agents for stimulating the synthesis of dermal or epidermal macromolecules and/or for preventing their degradation; agents for stimulating the proliferation of fibroblasts and/or keratinocytes or for stimulating keratinocyte differentiation; myorelaxants; antipollution agents and/or free-radical scavengers; slimming agents; agents that act on the capillary circulation; agents that act on the energy metabolism of cells; and mixtures thereof.

Thus, the composition and/or emulsion according to the invention may especially contain at least one active agent selected from the group consisting of: α-hydroxy acids; salicylic acid and its derivatives such as 5-n-octanoylsalicylic acid; HEPES; procysteine; O-octanoyl-6-D-maltose; the disodium salt of methyl glycine diacetic acid; ceramides; steroids such as diosgenin and DHEA derivatives; kojic acid; N-ethyloxycarbonyl-4-para-aminophenol; ascorbic acid and its derivatives; extracts of blueberry; retinoids and in particular retinol and its esters; polypeptides and acyl derivatives thereof; plant hormones; extracts of the yeast Saccharomyces cerevisiae; algal extracts; extracts of Vitreoscilla filiformis; extracts of soybean, lupin, corn and/or pea; alverine and its salts, in particular alverine citrate; resveratrol; carotenoids and in particular lycopene; tocopherol and its esters; coenzyme Q10 or ubiquinone; xanthines and in particular caffeine and natural extracts containing it; extracts of ruscus and of common horse chestnut; and mixtures thereof, without this list being limiting.

The composition and/or emulsion according to the invention may also contain at least one UVA and/or UVB screening agent. The sunscreens may be selected from the group consisting of organic screening agents and mineral screening agents, and mixtures thereof, for example.

As examples of UVA-active and/or UVB-active organic screening agents, mention may be made especially of the following, denoted hereinbelow by their CTFA name:

para-aminobenzoic acid derivatives: PABA, Ethyl PABA, Ethyl dihydroxypropyl PABA, Ethylhexyl dimethyl PABA sold in particular under the name “Escalol 507” by ISP, Glyceryl PABA, PEG-25 PABA sold under the name “Uvinul P25” by BASF,

salicylic derivatives: Homosalate sold under the name “Eusolex HMS” by Rona/EM Industries, Ethylhexyl salicylate sold under the name “Neo Heliopan OS” by Haarmann and Reimer, Dipropylene glycol salicylate sold under the name “Dipsal” by Scher, TEA salicylate sold under the name “Neo Heliopan TS” by Haarmann and Reimer,

dibenzoylmethane derivatives: Butyl methoxy-dibenzoylmethane sold in particular under the trade name “Parsol 1789” by Hoffmann LaRoche, Isopropyl-dibenzoylmethane,

cinnamic derivatives: Ethylhexyl methoxycinnamate sold in particular under the trade name “Parsol MCX” by Hoffmann LaRoche, Isopropyl methoxycinnamate, Isoamyl methoxycinnamate sold under the trade name “Neo Heliopan E 1000” by Haarmann and Reimer, Cinoxate, DEA methoxycinnamate, Diisopropyl methoxycinnamate, Glyceryl ethylhexanoate dimethoxycinnamate,

β,β′-diphenylacrylate derivatives: Octocrylene sold in particular under the trade name “Uvinul N539” by BASF, Etocrylene sold in particular under the trade name “Uvinul N35” by BASF,

benzophenone derivatives: Benzophenone-1 sold under the trade name “Uvinul 400” by BASF, Benzophenone-2 sold under the trade name “Uvinul D50” by BASF, Benzophenone-3 or Oxybenzone sold under the trade name “Uvinul M40” by BASF, Benzophenone-4 sold under the trade name “Uvinul MS40” by BASF, Benzophenone-5, Benzophenone-6 sold under the trade name “Helisorb 11” by Norquay, Benzophenone-8 sold under the trade name “Spectra-Sorb UV-24” by American Cyanamid, Benzophenone-9 sold under the trade name “Uvinul DS-49” by BASF, Benzophenone-12,

benzylidenecamphor derivatives: 3-Benzylidenecamphor, 4-Methylbenzylidenecamphor sold under the name “Eusolex 6300” by Merck, Benzylidenecamphorsulphonic acid, Camphor benzalkonium methosulphate, Terephthalylidenedicamphorsulphonic acid, Polyacrylamidomethylbenzylidenecamphor,

phenylbenzimidazole derivatives: Phenylbenzimidazolesulphonic acid sold in particular under the trade name “Eusolex 232” by Merck, benzimidazilate sold under the trade name “Neo Heliopan AP” by Haarmann and Reimer.

triazine derivatives: Anisotriazine sold under the trade name “Tinosorb S” by Ciba Geigy, Ethylhexyltriazone sold in particular under the trade name “Uvinul T150” by BASF, Diethylhexylbutamidotriazone sold under the trade name “Uvasorb HEB” by Sigma 3V,

phenylbenzotriazole derivatives: Drometrizole trisiloxane sold under the name “Silatrizole” by Rhodia Chimie,

anthranilic derivatives: Menthyl anthranilate sold under the trade name “Neo Heliopan MA” by Haarmann and Reimer,

imidazoline derivatives: Ethylhexyldimethoxybenzylidenedioxoimidazoline propionate,

benzalmalonate derivatives: Polyorganosiloxane containing benzalmalonate functions, sold under the trade name “Parsol SLX” by Hoffmann LaRoche,

and mixtures thereof.

The organic UV-screening agents that are more particularly preferred are selected from the group consisting of the following compounds:

Ethylhexyl salicylate,

Butylmethoxydibenzoylmethane,

Ethylhexyl methoxycinnamate,

Octocrylene,

Phenylbenzimidazolesulphonic acid,

Terephthalylidenedicamphorsulphonic acid,

Benzophenone-3,

Benzophenone-4,

Benzophenone-5,

4-Methylbenzylidenecamphor,

Benzimidazilate,

Anisotriazine,

Ethylhexyltriazone,

Diethylhexylbutamidotriazone,

Methylenebis(benzotriazolyl)tetramethylbutylphenol,

Drometrizole trisiloxane,

and mixtures thereof.

The mineral screening agents that can be used in the composition and/or emulsion according to the invention are in particular nanopigments (mean size of the primary particles: generally between 5 nm and 100 nm and preferably between 10 nm and 50 nm) of coated or uncoated metal oxides such as, for example, nanopigments of titanium oxide (amorphous or crystallized in rutile and/or anatase form), of iron oxide, of zinc oxide, of zirconium oxide or of cerium oxide. Coating agents are, moreover, alumina and/or aluminium stearate. Such coated or uncoated metal oxide nanopigments are described in particular in patent applications EP-A-0 518 772 and EP-A-0 518 773.

The compostion and/or emulsion according to the invention may also contain adjuvants that are common in cosmetics and dermatology, such as hydrophilic or lipophilic gelling agents, preserving agents, solvents, fragrances, fillers, pigments, odour absorbers and dyestuffs. The amounts of these various adjuvants are those conventionally used in the fields under consideration, and for example from 0.01% to 20% relative to the total weight. Depending on their nature, these adjuvants may be introduced into the fatty phase or into an aqueous phases. These adjuvants and the concentrations thereof should be such that they do not harm the advantageous properties of the tensioning agent.

The invention will now be illustrated by the following non-limiting examples. In these examples, the amounts are indicated as percentages by weight.

### EXAMPLES

### Example 1

### Cosmetic Composition

| | |
|---|---|
| Primary emulsion (A): | |
| Water: | 44.85 g |
| Polyglyceryl-4 isostearate, hexyl laurate and cetyl PEG/ | 3.50 g |
| PPG 10/1 dimethicone: | |
| Cyclopentasiloxane: | 16.50 g |
| Dimethicone: | 4.00 g |
| Colloidal silica: | 31.15 g |
| Multiple emulsion: | |
| Primary emulsion (A): | 22.50 g |
| Cyclopentasiloxane: | 3.50 g |
| Apricot kernel oil | 4.00 g |
| Water: | 68.05 g |
| Preserving agents: | 1.00 g |
| Pentasodium ethylenediaminetetramethylenephosphonate: | 0.05 g |
| Alkylacrylate copolymer: | 0.60 g |
| Sodium hydroxide: | 0.30 g |

Procedure

Preparation of the Primary Emulsion:

The Polyglyceryl-4 isostearate, the hexyl laurate and the cetyl PEG/PPG 10/1 dimethicone, the cyclopentasiloxane and the dimethicone are homogenized at room temperature and with stirring. The water and the colloidal silica are incorporated slowly with vigorous stirring.

Preparation of the Triple Emulsion:

The alkylacrylate copolymer, the preserving agents and the sequestering agent (pentasodium ethylenediaminetetramethylenephosphonate) are dispersed at room temperature and with stirring. The mixture is left to swell for about 45 minutes with stirring and is then neutralized with the sodium hydroxide. The primary emulsion is diluted with the cyclopentasiloxane and the apricot kernel oil, and this mixture is then incorporated slowly with stirring into the aqueous phase.

### Comparative Example 2

### W/O/W Multiple Emulsion Containing a Tensioning Agent in the Outer Aqueous Phase

| | |
|---|---|
| Primary emulsion (A): | |
| Water: | 75.20 g |
| Polyglyceryl-4 isostearate, hexyl laurate and cetyl PEG/ | 3.50 g |
| PPG 10/1 dimethicone: | |
| Cyclopentasiloxane: | 16.50 g |
| Dimethicone: | 4.00 g |
| Magnesium sulphate: | 0.80 g |
| Multiple emulsion: | |
| Primary emulsion (A): | 22.50 g |
| Cyclopentasiloxane: | 3.50 g |
| Apricot kernel oil | 4.00 g |
| Water: | 61.05 g |
| Preserving agents: | 1.00 g |
| Pentasodium ethylenediaminetetramethylenephosphonate: | 0.05 g |
| Alkylacrylate copolymer: | 0.60 g |
| Sodium hydroxide: | 0.30 g |
| Colloidal silica: | 7.00 g |

### Comparative Examples 3 and 4

### O/W Direct Emulsion and W/O Reverse Emulsion Containing a Tensioning Agent

The sense (W/O or O/W) of the emulsion is determined by the order of incorporation of the aqueous and oily phases. These emulsions are prepared in a manner that is standard for those skilled in the art.

| | |
|---|---|
| Oily phase: | |
| Polyglyceryl-4 isostearate, hexyl laurate and cetyl PEG/ | 0.79 g |
| PPG 10/1 dimethicone: | |
| Cyclopentasiloxane: | 7.21 g |
| Dimethicone: | 0.90 g |
| Apricot kernel oil: | 4.00 g |
| Aqueous phase: | |
| Water: | 78.15 g |
| Colloidal silica: | 7.00 g |
| Preserving agents: | 1.00 g |
| Pentasodium ethylenediaminetetramethylenephosphonate: | 0.05 g |
| Alkylacrylate copolymer: | 0.60 g |
| Sodium hydroxide: | 0.30 g |

### Example 5

### W/O/W Triple Emulsion Containing an IPN

| | |
|---|---|
| Multiple emulsion: | |
| Primary emulsion (A) | 22.50 g |
| Cyclopentasiloxane | 3.50 g |
| Apricot kernel oil | 4.00 g |
| Water | 58.05 g |
| Phenoxyethanol | 1.00 g |
| Pentasodium salt of ethylenediaminetetramethylenephos- | 0.05 g |
| phonate | |
| Acrylic acid/stearyl methacrylate copolymer (Pemulen TR1 | 0.60 g |
| from Noveon) | |
| Sodium hydroxide | 0.30 g |
| Anionic aqueous 40% dispersion of an interpenetrated net- | 10.00 g |
| work of polyurethane and polyacrylic polymers (Hybridur 875 | |
| from Air Products) | |
| Primary Emulsion (A): | |
| Water: | 75.20 g |
| Mixture of polyglyceryl isostearate (4 mol), hexyl laurate and | 3.50 g |
| oxyethylenated oxypropylenated polymethylcetyl dimethyl | |
| methylsiloxane (Abil WE09 from Goldschmidt) | |
| Cyclopentasiloxane | 16.50 g |
| Polydimethylsiloxane | 4.00 g |
| Magnesium sulphate | 0.80 g |

Procedure

Preparation of the Primary Emulsion:

The Abil WE09, the cyclopentasiloxane and the polydimethylsiloxane are homogenized at room temperature and with stirring. The water and the magnesium sulphate are incorporated slowly with vigorous stirring.

Preparation of the Multiple Emulsion:

The acrylic copolymer, the phenoxyethanol and the phosphonic sequestering agent are dispersed at room temperature and with stirring. The mixture is left to swell for about 45 minutes with stirring, followed by addition of the Hybridur 875. The resulting mixture is then neutralized with the sodium hydroxide, and the primary emulsion is then diluted with the cyclopentasiloxane and the apricot kernel oil. This mixture is then incorporated slowly with stirring into the aqueous phase.

### Example 6

### Demonstration of the Tensioning Effect

This example compares the tensioning effect obtained using the compositions of Examples 1 to 4 above, which all contain 7% colloidal silica as tensioning agent.

These compositions were tested using a dermometer. This machine was described by L. Rasseneur et al. in Influence des Différents Constituants de la Couche Cornée sur la Mesure de son Elasticité [Effect of Various Constituents of the Horny Layer on the Measurement of its Elasticity], International Journal of Cosmetic Science, 4, 247-260 (1982).

The principle of the method consists in measuring the length of a specimen of stratum corneum isolated from a human skin obtained from a surgical operation, before and after treatment with the test compositions.

To do this, the specimen is placed between the two jaws of the machine, one of which is fixed and the other mobile, in an atmosphere at 30° C. and 40% relative humidity.

A traction is exerted on the specimen, and the curve of the force (in grams) as a function of the length (in millimetres) is recorded, the zero length corresponding to the contact between the two jaws of the machine.

The tangent to the curve in its linear region is then plotted. The intersection of this tangent with the x-axis corresponds to the apparent length L₀ of the specimen at zero force.

The specimen is relaxed and 2 mg/cm² of the test composition are then applied to the stratum corneum. After drying for 15 minutes, the above steps are repeated to determine the length L₁ of the specimen after treatment.

The percentage of retraction is defined by:

% retraction=100×(L₁−L₀)/L₀

To characterize a tensioning effect, this percentage must be negative and the tensioning effect is proportionately greater the larger the absolute value of the percentage of retraction.

The results obtained are collated in Table 1 below:

TABLE 1

| | % of retraction of a specimen |
|---|---|
| Compositions | of isolated stratum corneum |
| Control composition (composition of | −0.23% ± 0.20 |
| Example 1 without silica) | |
| Example 1: W/O/W multiple emulsion | −1.18% ± 0.34 |
| containing 7% colloidal silica in the | |
| inner aqueous phase | |
| Example 2: W/O/W multiple emulsion | −0.69% ± 0.29 |
| containing 7% colloidal silica in the | |
| outer aqueous phase | |
| Example 3: O/W direct emulsion con- | −0.51% ± 0.29 |
| taining 7% colloidal silica | |
| Example 4: W/0 reverse emulsion con- | −0.70% ± 0.24 |
| taining 7% colloidal silica | |

It emerges from the above table that only the composition of Example 1 (composition according to the invention) has a higher retraction of the stratum corneum, as an absolute value, at 1%, when it is measured at 30° C. and under a relative humidity of 40%.

In particular, the retraction value obtained with the composition of Example 1 is significantly higher than that obtained with the composition of Example 2 (p<0.02).

Thus, the formulation of a tensioning agent such as colloidal silica in those systems described above (comprising an oily phase) other than the inner aqueous phase of a W/O/W emulsion do not make it possible to obtain a sufficient tensioning effect on the skin.

### Example 7

### Sensory Evaluation

The composition of Example 1 was applied on a panel of seven women with wrinkles and fine lines around the contour of the eyes. A smoothing-out effect of the fine lines under the eyes was observed after application of the composition.

The above written description of the invention provides a manner and process of making and using it such that any person skilled in this art is enabled to make and use the same, this enablement being provided in particular for the subject matter of the appended claims and including a composition comprising a W/O/W multiple emulsion comprising an inner aqueous phase, an oily phase and an outer aqueous phase, the emulsion containing at least one tensioning agent that produces, at a concentration of 7% in water, a retraction of isolated stratum corneum of more than 1% at 30° C. under a relative humidity of 40%, wherein the tensioning agent is present at least in the inner aqueous phase of the emulsion.

Preferred embodiments of the invention similarly fully described and enabled are a W/O/W multiple emulsion comprising an inner aqueous phase, an oily phase, and an outer aqueous phase, said emulsion comprising at least one tensioning agent, the tensioning agent being present at least in the inner aqueous phase of the emulsion, the tensioning agent being one that produces, at a concentration of 7% in water, a retraction of isolated stratum corneum of more than 1% at 30° C. under a relative humidity of 40%, a process for treating wrinkled skin, comprising applying the invention emulsion/composition to wrinkled skin, and a process for smoothing out wrinkles and fine lines and/or for restoring tautness to the skin, comprising applying the emulsion/composition of the invention to skin in need thereof. Similarly enabled is a composition comprising the invention emulsion, a preferred composition optionally further comprising an additional aqueous phase and/or additional oil phase, the composition optionally being in the form of a W/O/W/O emulsion. The uses of the composition are similarly enabled as for the invention emulsion.

All references, patents, applications, tests, standards, documents, publications, brochures, texts, articles, etc. mentioned herein are incorporated herein by reference. Where a numerical limit or range is stated, all values and subranges therewithin are specifically included as if explicitly written out.

What is claimed is:

1. A W/O/W multiple emulsion comprising an inner aqueous phase, an oily phase, and an outer aqueous phase, said emulsion comprising at least one tensioning agent, the tensioning agent being present at least in the inner aqueous phase of the emulsion, the tensioning agent being one that produces, at a concentration of 7% in water, a retraction of isolated stratum corneum of more than 1% at 30° C. under a relative humidity of 40%.

2. The emulsion according to claim 1, wherein the tensioning agent is selected from the group consisting of synthetic polymers, polymers of natural origin, plant proteins and protein hydrolysates, mixed silicates, wax microparticles, and colloidal particles of mineral fillers.

3. The emulsion according to claim 2, comprising a synthetic polymer selected from the group consisting of polyurethane polymers and copolymers, acrylic polymers and copolymers, sulphonated isophthalic acid polymers, and grafted silicone polymers.

4. The emulsion according to claim 2, comprising a synthetic polymer selected from the group consisting of networks of interpenetrated polymers.

5. The emulsion according to claim 4, wherein the interpenetrated polymer is added in the form of an aqueous dispersion of polyurethane and polyacrylic particles, with a weight-average size of between 90 and 110 nm, a number-average size of about 80 nm and a glass transition temperature, Tg, which ranges from about −60° C. to +100° C.

6. The emulsion according to claim 3, comprising a polydimethylsiloxane onto which are grafted, via a thiopropylene linking chain, poly(meth)acrylic acid and polyalkyl (meth)acrylate mixed polymer units.

7. The emulsion according to claim 2, comprising a polymer of natural origin selected from the group consisting of polyholosides and latexes.

8. The emulsion according to claim 7, comprising a starch selected from the group consisting of rice starch, corn starch, potato starch, cassava starch, pea starch, Triticum aestivum wheat starch and oat starch.

9. The emulsion according to claim 7, comprising a polyholoside selected from the group consisting of carrageenans, alginates, agars, gellans, cellulose-based polymers and pectins.

10. The emulsion according to claim 7, wherein the emulsion comprises a polyholoside, and wherein said polyholoside is added in the form of an aqueous dispersion of gel microparticles.

11. The emulsion according to claim 7, comprising a latex selected from the group consisting of shellac resin, sandarac gum, dammar resins, elemi gums, copal resins, cellulose-based derivatives, and mixtures thereof.

12. The emulsion according to claim 2, comprising a plant protein or protein hydrolysate selected from the group consisting of proteins and protein hydrolysates from corn, rye, Triticum aestivum wheat, buckwheat, sesame, spelt, pea, bean, lentil, soybean and lupin.

13. The emulsion according to claim 2, comprising a laponite.

14. The emulsion according to claim 2, comprising a wax selected from the group consisting of carnauba wax, candelilla wax and alfalfa wax.

15. The emulsion according to claim 2, comprising a mineral filler selected from the group consisting of silica, cerium oxide, zirconium oxide, alumina, calcium carbonate, barium sulphate, calcium sulphate, zinc oxide and titanium dioxide.

16. The emulsion according to claim 15, comprising silica.

17. The emulsion according to claim 2, wherein said colloidal particles have a diameter of between 3 and 30 nm.

18. The emulsion according to claim 15, wherein the outer aqueous phase is free of polyacrylamide gelling agent and triethanolamine.

19. The emulsion according to claim 1, wherein it is suitable for topical application to the skin.

20. The emulsion according to claim 1, wherein the tensioning agent is present in an amount of from 0.1% to 7% by weight relative to the total weight of the emulsion.

21. The emulsion according claim 1, wherein it is free of glycerol and of propylene glycol.

22. A process for treating wrinkled skin, comprising applying the emulsion of claim 1 to wrinkled skin.

23. A process for smoothing out wrinkles and fine lines and/or for restoring tautness to the skin, comprising applying the emulsion of claim 1 to skin in need thereof.

24. A composition comprising the emulsion of claim 1.

25. The composition according to claim 24, further comprising
an additional aqueous phase and/or oil phase.

26. The composition according to claim 25, further comprising
an additional oil phase.

27. The composition according to claim 26, wherein said
composition is a W/OIW/O emulsion.

28. A process for treating wrinkled skin, comprising applying the composition of claim 24 to wrinkled skin.

29. A process for smoothing out wrinkles and fine lines and/or for restoring tautness to the skin, comprising applying the composition of claim 24 to skin in need thereof.
